(19) United States
(12) Patent Application Publication
(10) Pub. No.: US 2011/0130833 A1
(43) Pub. Date: Jun. 2, 2011

(54) INTRAOCULAR LENS HAVING EDGE CONFIGURED TO REDUCE POSTERIOR CAPSULE OPACIFICATION

(76) Inventors: James M. Scott, Millsap, TX (US); Huong Huynh, Arlington, TX (US); Stephen J. Van Noy, Southlake, TX (US); William M. Graham, Kenmore, WA (US)

(21) Appl. No.: 12/951,869

(22) Filed: Nov. 22, 2010

Related U.S. Application Data
(60) Provisional application No. 61/265,469, filed on Dec. 1, 2009.

(51) Int. Cl. A61F 2/16

(52) U.S. Cl. 623/6.49

(57) ABSTRACT

An intraocular lens (IOL) for implantation within a capsular bag includes an optic and a plurality of haptics. The optics has an anterior optic face and a posterior optic face joined by a peripheral wall. The peripheral wall includes a straight portion of uniform width extending posteriorly from the anterior optic face to a flare point and a flared optic edge. The flared optic edge extends posteriorly and widens from the flare point and meets the posterior optic face at a sharp optic corner. Each of the haptics is coupled to the optic at the peripheral wall at respective haptic-optic junctions. The flared optic edge surrounds the peripheral wall between the haptic-optic junctions.

### RELATED APPLICATIONS

This application claims priority to U.S. provisional application Ser. No. 61/265,469, filed on Dec. 1, 2009, the contents which are incorporated herein by reference.

### FIELD OF THE INVENTION

This invention relates to intraocular lenses (IOIs) and more particularly to an IOL having a edge configured to reduce posterior capsule opacification.

### BACKGROUND OF THE INVENTION

The human eye in its simplest terms functions to provide vision by transmitting and refracting light through a clear outer portion called the cornea, and further focusing the image by way of the lens onto the retina, at the back of the eye. The quality of the focused image depends on many factors including the size, shape and length of the eye, and the shape and transparency of the cornea and lens. When trauma, age or disease cause the lens to become less transparent, vision deteriorates because of the diminished light which can be transmitted to the retina. This deficiency in the lens of the eye is medically known as a cataract. The treatment for this condition is surgical removal of the lens and implantation of an artificial intraocular lens (“IOL”). An IOL is generally implanted in the capsular bag by formation of an anterior capsulorhexis that leaves a capsular bag remnant including the posterior wall of the capsule and an anterior “leaflet” surrounding the capsulorhexis.

One complication that can arise with the implantation of IOLs in the capsular bag is that capsular cells can grow around or on the IOL in such a way that the capsular bag remnant becomes opaque, a phenomenon known as posterior capsule opacification (“PCO”). Correction of PCO often requires subsequent surgical intervention using an Nd/YAG laser to remove the opaque regions of the posterior capsule wall. While various techniques have been employed to help reduce this phenomenon, such as placing corners at the edges of the optic contacting the wall and pressing the IOL against the capsular bag to keep the capsular wall taut, undesired growth of capsular cells remains problematic for IOLs

### BRIEF SUMMARY OF THE INVENTION

In particular embodiments of the present invention, an intraocular lens (IOL) for implantation within a capsular bag includes an optic and a plurality of haptics. The optic has an anterior optic face and a posterior optic face joined by a peripheral wall. The peripheral wall includes a straight portion of uniform width extending posteriorly from the anterior optic face to a flare point and a flared optic edge. The flared optic edge extends posteriorly and widens from the flare point and meets the posterior optic face at a sharp optic corner. Each of the haptics is coupled to the optic at the peripheral wall at respective haptic-optic junctions. The flared optic edge surrounds the peripheral wall between the haptic-optic junctions.

In particular embodiments of the present invention, a method of manufacturing an IOL includes forming a circular mold for a pre-milled IOL; molding a refractive material in the circular mold to form the pre-milled IOL; and removing the refractive material from the pre-milled IOL to form an IOL. The IOL includes haptics and an optic. The optic has an anterior optic face and a posterior optic face joined by a peripheral wall. The peripheral wall includes a straight portion of uniform width extending posteriorly from the anterior optic face to a flare point and a flared optic edge. The flared optic edge extends posteriorly and widens from the flare point and meets the posterior optic face at a sharp optic corner. Each of the haptics is coupled to the optic at the peripheral wall at respective haptic-optic junctions. The flared optic edge surrounds the peripheral wall between the haptic-optic junctions.

Other objects, features and advantages of the present invention will become apparent with reference to the drawings, and the following description of the drawings and claims.

### BRIEF DESCRIPTION OF THE DRAWINGS

FIGS. 1A and 1B illustrate an intraocular lens (IOL) according to a particular embodiment of the present invention;

FIG. 2 illustrates a flared edge according to particular embodiments of the present invention in detail;

FIG. 3 illustrates examples of a flared edge according to particular embodiments of the present invention in further detail; and

FIG. 4 is a flow chart showing an example method for manufacturing an IOL according to particular embodiments of the present invention.

### DETAILED DESCRIPTION OF THE INVENTION

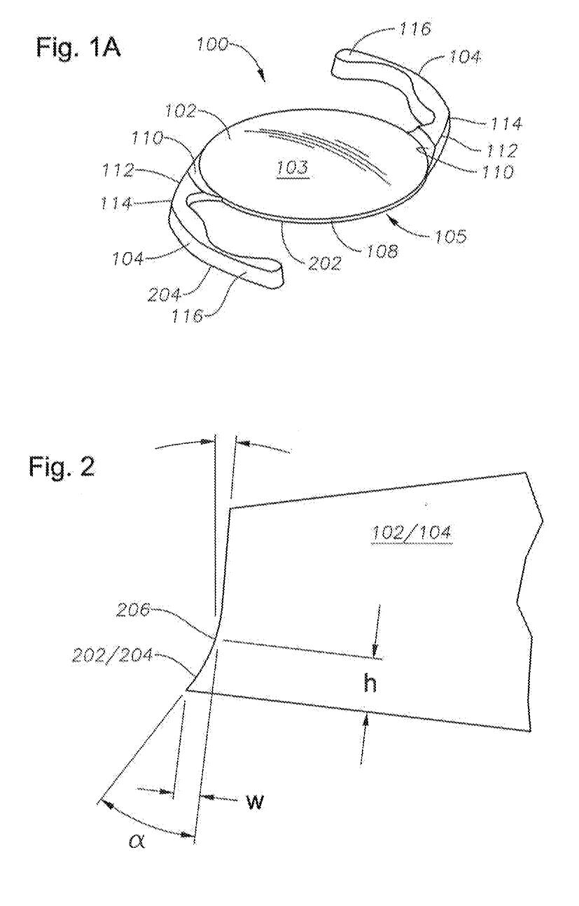

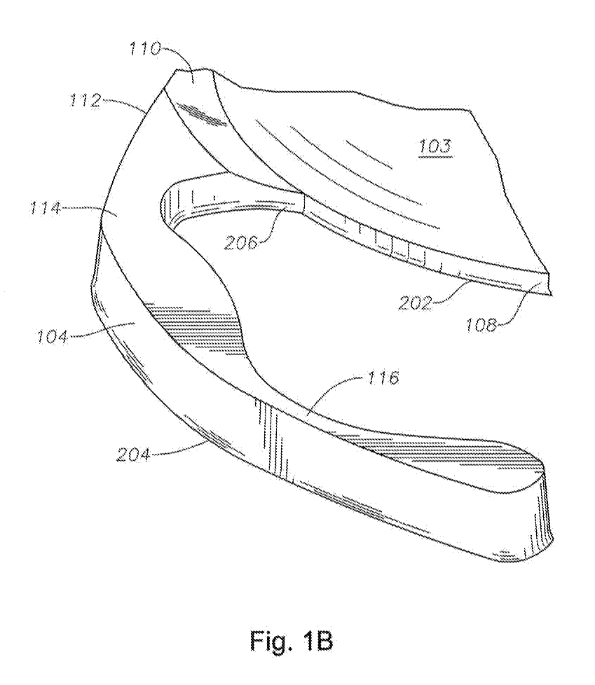

FIG. 1A illustrates an intraocular lens (IOL) 100 suitable for implantation within a capsular bag of an eye according to a particular embodiment of the present invention. (FIG. 1B shows a magnified view of the section of the IOL 100 within the dashed box of FIG. 1A.) The IOL 100 includes an optic 102, referring to a central generally circular body that includes the optical region configured focus light onto the retina, and flexible haptics 104 that serve to position the IOL 100 within the capsular bag remnant following capsulorhexis. The optic 102 has an anterior optic face 103 and a posterior optic face 105 that are joined by a peripheral wall 108. The peripheral wall 108 includes a straight portion 109 having a uniform radial width extending posteriorly from the anterior optic face 103. The peripheral wall 108 further includes a flared edge 202 that meets the straight portion at a flare point 206, at which point the radial width of the peripheral wall 108 begins to continuously increase in the posterior direction.

The haptics 104 are joined to the peripheral wall 108 at haptic-optic junctions 110. In the depicted embodiment, at the haptic-optic junction 110 at the anterior face 103 of the optic 102, the haptics 104 have a ramp where the thickness increases from the thickness of the optic 102 to the thickness of the majority of the haptic 104. In this context and more generally for purposes of this specification, the term “thickness” refers to a thickness measured in the anterior-to-posterior direction, and comparative terms such as “less than” or “greater than” refer to a thickness of a particular feature remaining within that range through its entire range of extension, such as the haptics 104 of the depicted embodiment in FIG. 1 having a thickness that is more than the thickness of the optic.

The haptics 104 as depicted include a proximal portion 112 extending from the optic 102 to a flexible joint 114 and a distal contact portion 116 that contacts the capsular bag. The proximal and distal portions 112 and 116 each have anterior and posterior faces and lateral sides. For purposes of this specification, “lateral” refers to a direction perpendicular both to the optical axis and to a direction of the haptic's extension outwardly from the optic. Near the optic 102, the haptics 104 also include gussets of increased lateral width to help in maintaining the mechanical stability of the haptics 104. Various considerations regarding the structure and function of haptics in general that can be employed in conjunction with IOLs according to particular embodiments of the present invention are also discussed in U.S. Pat. No. 5,716,403 to Tran et al., which is incorporated herein by reference.

In particular embodiments, the IOL 100 may be formed entirely from a refractive material. Examples of suitable refractive materials include acrylics, hydrogels, and silicone; other suitable materials for foldable IOLs will be well known to those skilled in the art. It may be desirable for the flared edge 202 to maintain sufficient mechanical rigidity to avoid deformation by the capsular bag. However, as discussed in detail below, it is also possible for the flared edge 202 to function to reduce PCO even if the flared edge 202 is deformable, so long as a sharp corner of the flared edge 202 is in contact with the capsular bag. All or part of the IOL 100 may include a coating or other material that acts to deter PCO through biological or chemical action as well. Various such coatings ic and/or materials are known to those skilled in the art.

As the posterior side of the IOL 100 presents a flat, smooth surface to the capsular bag, there is some possibility that capsular cell growth beginning at one point on the surface may progressively extend to other portions, including the visual field of the optic 102. Conventional techniques use a square corner surrounding the edge of the optic 102, so as to provide a sharp corner in contact with the capsular bag, but if there is no offset between the haptics 104 and the optic 102, the haptic-optic junction 110 remains smooth, which could conceivably provide a path for capsular cell migration. To correct that problem of a continuous path between the haptics and the optic, there are previously known techniques for placing a pointed edge extending in the posterior direction from the optic, so that the edge surrounds the entire optic including the haptic-optic junctions. However, such edges present manufacturing difficulties, and the creation of a wall around the optic can potentially exacerbate capsular cell growth by providing a contained area for capsular cells to grow on the optic.

As contrasted with prior techniques, various embodiments of the present invention provide a pointed edge that does not extend posteriorly. Instead, flared edges according to particular embodiments of the present invention form a point directed generally within the plane of the optic around the optic and/or laterally from the haptics. Thus, for example, the IOL 100 depicted in FIG. 1 includes a flared optic edge 202 and flared haptic edges 204. The flared haptic edges 204 are shown extending around the entire haptic, but the flared haptic edges 204 can also extend partially around the haptics 104. For example, the flared haptic edges 204 may extend only around an outer side of the distal contact portion 116 of the haptics 104. This allows a sharp corner of the flared edges 202 and/or 204 to be placed in contact with the capsular bag without the corner needing to extend posteriorly toward the capsular bag.

As shown in detail in FIG. 2, the flared edges 202 and 204 are continuously widening portions of the optic 102 and haptics 104, respectively, which meet the posterior faces of the optic 102 or haptic 104 at a sharp corner. This produces a sharp corner that is pointed in a direction at least 90 degrees, and perhaps slightly more, away from the posterior wall of the capsular bag. The flared optic edges 202 increase continuously from a first radial width value to a second radial width value, shown as width w, and may widen according to a particular flare angle a, which may be, for example, from 5 to 10 degrees. The straight portion anterior of the flare point 206 may be angled as well, with the flare angle being with respect to the straight portion. Likewise, the flared haptic edges 204 increase from a first lateral width value to a second lateral width value. The flared edges 202 and 204 have respective heights h measured along the thickness of the optic 102 or haptics 104 measured from the posterior face of the optic 102 or haptic 104 to a flare point 206 at which the continuous increase in width begins. According to various embodiments of the present invention, the height of the flared edges 202 and 204 is less than half of the thickness of the optic 102 or haptic 104, respectively.

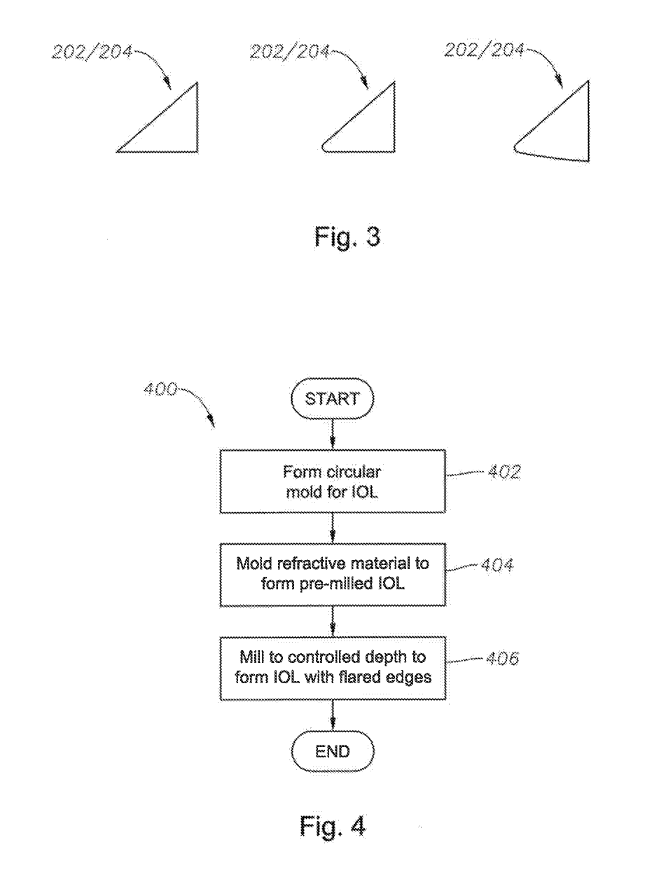

As compared to manufacturing the more complicated edge structures that point posteriorly, manufacturing the flared edges 202 and 204 may be less complex. Several options for forming the shape of the flared edges 202 or 204 around the optic 102 and haptics 104 may be available, so that, for example, the flared edges 202 or 204 may have a corner or a rounded bump at the flare point to account for manufacturing tolerances, and the underlying surface may be radiused as well. These examples are all illustrated in FIG. 3. Likewise, the height and flare angle of the flared edges 202 and 204 can be selected in combination with manufacturing tolerances to produce a suitably sharp and uniform corner with sufficient mechanical rigidity to resist deformation by the capsular bag. Various embodiments of the present invention are amenable to forming the IOLs using a variety of known manufacturing techniques, including molding and/or milling.

FIG. 4 is a flow chart 400 showing the steps of an example method for manufacturing an IOL 100 according to particular embodiments of the present invention. At step 402, a circular mold for the IOL 100 is formed. The circular mold includes the forms for the anterior optic face 104 and the posterior optic face 106. At step 404, the refractive material of the IOL 100 is molded in the circular mold, which forms the optic 102 surrounded by a circular region of molded material referred to as a “pre-milled IOL.” At step 406, the pre-milled IOL is milled to define the edges of the peripheral wall 108 of the optic 102 and the lateral sides of the haptics 104. The milling is performed to a controlled depth so as to leave the flared edges 202 and 204 of the optics 102 and haptics 104 intact. This results in a completed IOL 100 according to various embodiments of the present invention. It should be understood, however, that other methods of material removal (e.g., etching) may be used, and more generally, other techniques of forming the flared edges 202 and 204, such as direct formation into the mold, may be employed consistent with various embodiments of the present invention.

The present invention is illustrated herein by example, and various modifications may be made by a person of ordinary skill in the art. Although the present invention is described in detail, it should be understood that various changes, substitutions and alterations can be made hereto without departing from the scope of the invention as claimed

What is claimed is:

1. An intraocular lens (IOL) for implantation within a capsular bag, comprising:
an optic having an anterior optic face and a posterior optic face joined by a peripheral wall, the peripheral wall comprising a straight portion of uniform width extending posteriorly from the anterior optic face to a flare point and further comprising a flared optic edge, the flared optic edge extending posteriorly and widening from the flare point and meeting the posterior optic face at a sharp optic corner; and
a plurality of haptics, each of the haptics being coupled to the optic at the peripheral wall at respective haptic-optic junctions, wherein the flared optic edge surrounds the peripheral wall between the haptic-optic junctions.

2. The IOL of claim 1, wherein the flared optic edge has a height less than half of a thickness of the peripheral wall.

3. The IOL of claim 1, further comprising a plurality of flared haptic edges, each of the haptic edges extending posteriorly along a respective lateral side of one of the haptics and widening from a flare point, each flared haptic edge meeting a posterior face of the respective haptic at a sharp haptic corner, the sharp haptic corner being sufficiently rigid to resist deformation by the capsular bag.

4. The IOL of claim 3, wherein the flared haptic edges extend at least along an outer lateral side of a distal contact portion of each of the haptics.

5. The IOL of claim 3, wherein the flared haptic edges entirely surround the haptics.

6. The IOL of claim 3, wherein each of the flared haptic edges has a height less than half of a thickness of the respective haptic.

7. The IOL of claim 1, wherein the flared optic edge has a flare angle between 5 and 10 degrees.

8. The IOL of claim 1, wherein the sharp optic corner is sufficiently rigid to resist deformation by the capsular bag.

9. A method of manufacturing an intraocular lens (IOL), comprising:
forming a circular mold for a pre-milled IOL;
molding a refractive material in the circular mold to form the pre-milled IOL; and
removing the refractive material from the pre-milled IOL to form an IOL, the IOL comprising:
an anterior optic face and a posterior optic face joined by a peripheral wall, the peripheral wall comprising a straight portion of uniform width extending posteriorly from the anterior optic face to a flare point and further comprising a flared optic edge, the flared optic edge extending posteriorly and widening from the flare point and meeting the posterior optic face at a sharp optic corner, the sharp optic corner being sufficiently rigid to resist deformation by the capsular bag; and
a plurality of haptics, each of the haptics being coupled to the optic at the peripheral wall at respective haptic-optic junctions, wherein the flared optic edge surrounds the peripheral wall between the haptic-optic junctions.

10. The method of claim 9, wherein the flared optic edge has a height less than half of a thickness of the peripheral wall.

11. The method of claim 9, wherein the step of removing the refractive material further comprises forming a plurality of flared haptic edges, each of the haptic edges extending posteriorly along a respective lateral side of one of the haptics and widening from a flare point, each flared haptic edge meeting a posterior face of the respective haptic at a sharp haptic corner, the sharp haptic corner being sufficiently rigid to resist deformation by the capsular bag.

12. The method of claim 11, wherein the flared haptic edges extend at least along an outer lateral side of a distal contact portion of each of the haptics.

13. The method of claim 11, wherein the flared haptic edges entirely surround the haptics.

14. The method of claim 11, wherein each of the flared haptic edges has a height less than half of a thickness of the respective haptic.

15. The method of claim 9, wherein the flared optic edge has a flare angle between 5 and 10 degrees.
